(19) United States
(12) Patent Application Publication
(10) Pub. No.: US 2008/0293631 A1
(43) Pub. Date: Nov. 27, 2008

(54) Treatment for Autoimmune and Inflammatory Conditions

(76) Inventors: Christopher John Jackson, Mt. Colah (AU); Meilang Xue, Campsie (AU)

Correspondence Address: CLARK & ELBING LLP, 101 FEDERAL STREET, BOSTON, MA, 02110, US

(21) Appl. No.: 11/813,506

(22) PCT Filed: Jan. 9, 2006
(86) PCT No.: PCT/AU06/00009

§ 371 Date: May 6, 2008

(30) Foreign Application Priority Data
Jan. 7, 2005 (AU) 2005900073

(51) Int. Cl. A61K 38/16; A61K 31/7088; A61P 19/02; A61P 3/10

(52) U.S. Cl. 514/12; 514/44

(57) ABSTRACT

The present invention provides methods for preventing the elevation of blood glucose levels, for preventing or delaying the onset of autoimmune and inflammatory diseases or glucose-associated conditions, and for preventing or reducing inflammation in individuals suffering from an autoimmune or inflammatory diseases, the methods comprising administering to subjects in need thereof an effective amount of activated Protein C or precursor Protein C.

### TECHNICAL FIELD

The present invention relates generally to methods and compositions for preventing the elevation of blood glucose levels and for the prevention of glucose-related conditions, including diabetes, and other autoimmune and inflammatory conditions, including rheumatoid arthritis.

### BACKGROUND OF THE INVENTION

Autoimmune and inflammatory diseases and disorders have debilitating effects of sufferers. For example, diabetes mellitus is a chronic autoimmune disease that requires long-term medical attention both to limit the development of its devastating complications and to manage such complications when they occur. It is a disproportionately expensive disease to treat; patients diagnosed with diabetes accounted for 5.8% of the US population in 1997, or 15.7 million people, but their per capita health care cost was $10,071, while it was $2,699 for those without diabetes.

Diabetes is widely recognised as one of the leading causes of death and disability in the world and is Australia's fastest growing chronic disease with almost 1500 people diagnosed every week. The morbidity and mortality associated with diabetes are related to the short- and long-term complications. These complications include hypoglycemia and hyperglycemia, increased risk of infections, microvascular complications (i.e., retinopathy, nephropathy), neuropathic complications, and macrovascular disease. Diabetes is the major cause of blindness in adults aged 20-74 years, as well as the leading cause of nontraumatic lower-extremity amputation and end-stage renal disease (ESRD).

Diabetes mellitus is traditionally divided into two types, type I (insulin-dependent) and type II (non insulin-dependent), both of which are typically characterized, at least in part, by elevated blood glucose levels. Type I diabetes accounts for up to 15% of all diabetes and is increasing in incidence especially in children less than five years of age. It is one of the most common chronic, life-long disorders beginning in children and is associated with serious vascular complications which pose a major health problem with considerable cost to the community, and account for almost 70% of deaths in diabetic patients. Type II diabetes is becoming increasingly common as average lifespans increase. Although generally associated with adult onset, type II diabetes is also being seen more frequently in younger people in association with the rising prevalence of childhood obesity.

The hallmark of type I diabetes is the selective destruction of insulin-producing cells in the pancreas, known as insulitis. Examination of islet tissue obtained from pancreatic biopsy from patients with recent onset type I diabetes confirms insulitis, with the presence of an infiltrate composed of CD4 and CD8 T lymphocytes, B lymphocytes, and macrophages, suggesting that these cells have a role in destruction of the cells.

Type 1 diabetes is considered primarily a T cell mediated disease. The onset and progression of type I diabetes is associated with dysregulated humoral and cellular immunity (Atkinson and Maclaren, 1994; Huck et al. 2001). Cell mediated immunity is thought to play a major role in the destructive islet inflammation that leads to selective β-cell damage. The initial interaction of genes and environmental factors seem to trigger an immune mediated response, with the appearance of autoantibodies as the first sign of cell destruction, followed eventually by the loss of the first phase insulin response. The progression to overt diabetes resulting in significant cell destruction is triggered by the development of a more aggressive T cell phenotype.

Whereas blood glucose levels in type I diabetes become elevated as a result of a lack of insulin production, in type II diabetes insulin production is not reduced, but rather there is a reduction in the response to insulin, so-called insulin resistance, by fat and muscle cells. To compensate for this reduced response, the pancreas increases insulin production. Insulin resistance may lead to type II diabetes when the pancreas fails to sustain the required increase in insulin production required to compensate for the body's reduced ability to respond to insulin activity.

No agent tested to date has proven to be efficacious in preventing the onset of diabetes in humans. Two major trials have been conducted in attempts to prevent type I diabetes. In the United States, a diabetes prevention trial was started in 1994 with the aim of determining whether antigen based treatment with insulin (oral and parenteral insulin treatment in relatives at high and moderate risk) would prevent or delay diabetes. These treatments did not slow the overall progression to diabetes (P Bingley, European Association of the Study of Diabetes, Budapest, September 2002). The European Nicotinamide Diabetes Intervention Trial also found no difference in protection from diabetes when participants were assigned to either oral nicotinamide or placebo treatment (Gale et al., 2004).

There is a clear need for the development of effective strategies and treatments to prevent the onset and development of diabetes.

Another inflammatory disease for which existing treatments have been found wanting is rheumatoid arthritis. In rheumatoid arthritis, the severity of joint inflammation and damage correlates with the degree of leukocyte infiltration of synovial tissue. Activated leukocytes secrete various inflammatory cytokines and proteases, including gelatinases such as matrix metalloproteinase (MMP)-9, which are important in initiating, propagating and maintaining the synovial inflammation in rheumatoid arthritis. Monocytes, one type of leukocyte, that migrate into tissues to become residential macrophages can also differentiate into dendritic cells and osteoclasts, the latter being recognized as the key cellular effectors of pathologic bone erosion in arthritis, MMP-9 is produced mainly by leukocytes, including neutrophils and monocytes/macrophages and is thought to play a key role in promoting invasion of these cells in rheumatoid arthritis (Jovanovic et al. 2000). MMP-9 is markedly elevated in serum and joints of rheumatoid arthritis patients and its levels correlate with the severity of rheumatoid arthritis (Giannelli et al., 2004).

The present invention is predicated in part on the inventors' surprising finding that in vivo administration of activated Protein C lowers blood glucose levels and prevents the onset of diabetes in a mouse model, and also reduces the incidence and severity of arthritis in a mouse model.

### SUMMARY OF THE INVENTION

According to a first aspect of the present invention there is provided a method for preventing the elevation of blood glucose levels in a subject, the method comprising administering to the subject an effective amount of activated Protein C or precursor Protein C.

The Protein C may be a polypeptide comprising the amino acid sequence as set forth in SEQ ID No. 1.

The Protein C may be administered in the form of a nucleic acid molecule encoding Protein C. The nucleic acid may comprise a nucleotide sequence as set forth in SEQ ID No. 2.

The nucleotide sequence may be located in a nucleic acid construct operably linked to a promoter active in the subject to be treated. The nucleic acid construct may be a DNA construct. The DNA construct may be a plasmid.

According to a second aspect of the present invention there is provided a method for preventing or delaying the onset of an autoimmune or inflammatory disease in a subject, the method comprising administering to the subject an effective amount of activated Protein C or precursor Protein C.

The autoimmune or inflammatory disease may be selected from the group consisting of: hyperglycaemia, postprandial hyperglycaemia, insulin resistance, type I diabetes, type II diabetes, and gestational diabetes. The autoimmune or inflammatory disease may be a disease of the musculoskeletal system, the nervous system, the gastrointestinal system, the blood or blood vessels, the endocrine system or the skin. The autoimmune or inflammatory disease may be arthritis, such as rheumatoid arthritis.

According to a third aspect of the present invention there is provided a method for preventing or reducing inflammation in a subject suffering from an autoimmune or inflammatory disease the method comprising administering to the subject an effective amount of activated Protein C or precursor Protein C.

The autoimmune or inflammatory disease may be arthritis, such as rheumatoid arthritis.

According to a fourth aspect of the present invention there is provided a method for preventing or delaying the onset of a glucose-associated condition in a subject, the method comprising administering to the subject an effective amount of activated Protein C or precursor Protein C. The glucose-associated condition may be selected from the group consisting of: hyperglycaemia, postprandial hyperglycaemia, impaired glucose tolerance, insulin resistance, type I diabetes, type II diabetes, and gestational diabetes.

According to a fifth aspect of the present invention there is provided a method for preventing or delaying the onset of diabetes in a subject, the method comprising administering to the subject an effective amount of activated Protein C or precursor Protein C.

The diabetes may be type I diabetes, type II diabetes or gestational diabetes.

According to a sixth aspect of the present invention there is provided a method for inhibiting the migration of leukocytes in a subject, the method comprising administering to the subject an effective amount of activated Protein C or precursor Protein C.

The leukocytes may be neutrophils, monocytes or lymphocytes.

According to any one of the above aspects and embodiments the protein or nucleic acid construct may be administered in the form of a composition together with one or more pharmaceutically acceptable carriers, adjuvants or diluents.

According to a seventh aspect of the present invention there is provided a composition when used for preventing the elevation of blood glucose levels, the composition comprising activated Protein C or precursor Protein C together with one or more pharmaceutically acceptable carriers, diluents or adjuvants.

According to an eighth aspect of the present invention there is provided a composition when used for preventing or delaying the onset of an autoimmune or inflammatory disease, the composition comprising activated Protein C or precursor Protein C together with one or more pharmaceutically acceptable carriers, diluents or adjuvants.

According to a ninth aspect of the present invention there is provided a composition when used for preventing or delaying the onset of a glucose-associated condition, the composition comprising activated Protein C or precursor Protein C together with one or more pharmaceutically acceptable carriers, diluents or adjuvants.

According to a tenth aspect of the present invention there is provided a composition when used for preventing or delaying the onset of diabetes, the composition comprising activated Protein C or precursor Protein C together with one or more pharmaceutically acceptable carriers, diluents or adjuvants.

According to an eleventh aspect of the present invention there is provided a composition when used for inhibiting migration of leukocytes, the composition comprising activated Protein C or precursor Protein C together with one or more pharmaceutically acceptable carriers, diluents or adjuvants.

According to the seventh to the eleventh aspects, the composition may be administered, for example, intranasally or orally. Alternatively, administration may be, for example, topical, intracavitary, subcutaneous, intramuscular, or intravenous.

According to a twelfth aspect of the present invention there is provided the use of activated Protein C or precursor Protein C for the manufacture of a medicament for the prevention of elevation of blood glucose levels in a subject.

According to a thirteenth aspect of the present invention there is provided the use of activated Protein C or precursor Protein C for the manufacture of a medicament for preventing or delaying the onset of an autoimmune or inflammatory disease in a subject.

According to a fourteenth aspect of the present invention there is provided the use of activated Protein C or precursor Protein C for the manufacture of a medicament for preventing or delaying the onset of a glucose-associated condition.

According to a fifteenth aspect of the present invention there is provided the use of activated Protein C or precursor Protein C for the manufacture of a medicament for preventing or delaying the onset of diabetes.

Typically, according to the above aspects and embodiments the subject is a human.

### DEFINITIONS

In the context of this specification, the term “comprising” means “including principally, but not necessarily solely”. Furthermore, variations of the word “comprising”, such as “comprise” and “comprises”, have correspondingly varied meanings.

As used herein the term “Protein C” refers to activated Protein C and precursors or derivatives thereof. Also encompassed within the scope of the invention are homologues or mimetics of activated Protein C which possess qualitative biological activity in common with the full-length mature activated Protein C. Further, the present invention contemplates not only use of the Protein C polypeptide, but also polynucleotides encoding the same.

The term “glucose-associated condition” as used herein refers to a disorder or condition associated with, at least in part, the accumulation of excess or abnormal glucose in the blood and/or a failure to reduce glucose levels in the blood to a physiological acceptable level. A “glucose-associated condition” may be characterized by the accumulation of blood glucose or failure to reduce blood glucose levels, may occur as a result (either directly or indirectly) of such accumulation or failure of reduction, or itself lead to the accumulation of glucose in the blood. One exemplary glucose-associated condition is diabetes. Accordingly, “glucose-associated condition” therefore also includes within its scope diabetes-related complications.

As used herein, “prevent” and “preventing” refers to the prevention, inhibition or retardation of the onset or development of a condition. For example in the case of diabetes, “preventing” may refer to the prevention of onset of diabetes or the prevention of the further development of the disorder once initiated.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein the term “polypeptide” means a polymer made up of amino acids linked together by peptide bonds.

As used herein, the term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

### BRIEF DESCRIPTION OF THE DRAWINGS

The present invention will now be described, by way of example only, with reference to the following drawings:

FIG. 1. Mean blood glucose levels of control and APC treated NOD mice from week 6 to week 25. Data are expressed as mean ±SEM of three independent experiments (total of 18 mice for each group).

FIG. 2. Incidence of diabetes in NOD mice following APC treatment from week 11 to week 25. Data are expressed as mean ±SEM of three independent experiments.

FIG. 3. Representative zymogram showing MMP-2 and MMP-9 secretion and activation in splenic T cells from NOD mice treated for 24 hours with 1, 5, 10 and 20 μg/ml APC.

FIG. 4. Effect of APC on the migration of A) neutrophils and B) monocytes from type I diabetic patients. The combination of IL-8 or MCP-1 with APC (10 μg/ml) showed decreased chemotactic activity for neutrophils and monocytes. *p<0.05, **p<0.01.

FIG. 5. The effect of APC on MMP-9 expression in monocytes from human RA patients. A. MMP-9 in the culture supernatants of RA monocytes treated with APC (0.0.1, 1, 10, 20 μg/ml) for 24 hours was detected by ELISA. Data represent mean ±SEM (n=3), *P<0.05, **P<0.01. B. Gene expression of MMP-9 detected by real time PCR in Mac6 cells treated with APC (20 μg/ml), lipopolysaccharide (LPS, 100 ng/ml), TNF-α (100 ng/ml) or APC plus LPS or TNF-α for 2 hours, Data represent mean ±SEM (n=3), *P<0.05.

FIG. 6. The effect of APC on NF-κB activation in monocytes from RA patients. The active form of NF-κB in whole cell lysates was detected by western blotting (A, B) using an antibody against active NF-κB. A. RA monocytes were treated with APC (20 μg/ml), LPS (100 ng/ml), LPS plus APC (20 μg/ml). B. RA monocytes treated LPS (100 ng/ml), LPS plus APC (20 μg/ml) either with RCR252 (a blocking antibody to EPCR) or with RCR92 (a non-blocking antibody to EPCR). All experiments were repeated at least twice.

FIG. 7. Effect of APC on the incidence and severity of collagen-induced arthritis in mice treated with APC or PBS (control). A. Average clinical score for each group every 2 days between day 7 and day 17 after second collagen boosting. P-values were obtained using a paired t-test, comparing APC- and PBS-treated mice.

The amino acid sequence set forth in SEQ ID NO:1 is the amino acid sequence of human Protein C.

The nucleotide sequence set forth in SEQ ID NO:2 is the nucleotide sequence of the gene encoding the human Protein C.

### BEST MODE OF PERFORMING THE INVENTION

Activated protein C (APC) is a serine protease having a molecular weight of about 56 kD that plays a central role in physiological anticoagulation. The inactive precursor, protein C, is a vitamin K-dependent glycoprotein synthesised by the liver and endothelium and is found in plasma. Activation of protein C occurs on the endothelial cell surface and is triggered by a complex formed between thrombin and thrombomodulin (Esmon et al., 1997). Another endothelial specific membrane protein, endothelial protein C receptor (EPCR), has been shown to accelerate this reaction more than 1000-fold. Endothelial APC functions as an anticoagulant by binding to the cofactor, protein S, on the endothelial surface, which inactivates the clotting factors Factor VIIIa and Factor Va.

The present inventors have previously found that APC is useful for the treatment of wounds and, particularly, slow-healing wounds by virtue of its ability to promote regeneration of endothelial cells after wounding in vitro and stimulate re-epithelialisation, fibroblast invasion and angiogenesis (WO 02/100445 the disclosure of which is incorporated herein by reference). Predicated on the surprising findings of the inventors as disclosed herein, the present invention relates to novel therapeutic and preventative uses for APC.

As disclosed herein, the inventors have now shown that APC prevents the elevation of blood glucose levels and prevents the onset of hyperglycaemia and diabetes.

Accordingly, one aspect of the present invention relates to a method for reducing blood glucose levels in a subject, the method comprising administering to the subject an effective amount of APC or precursor Protein C.

The present inventors have also found that APC inhibits the migration of leukocytes (monocytes and neutrophils) isolated from human type I diabetic patients. Leukocytes are the major cell type implicated in the pathogenesis of type I diabetes. Further, the inventors herein provide the first report on the effects of APC in inflammatory arthritis. The inventors have surprisingly shown that APC attenuates gene and protein expression of MMP-9 and inhibits the expression and activation of NF-κB in monocytes purified from the blood of RA patients and in Mac6 cells. The administration of APC is also shown to reduce the severity and incidence of arthritic symptoms in a mouse model of inflammatory (collagen-induced) arthritis.

Accordingly, aspects of the present invention also relate to methods for preventing or delaying the onset of glucose-associated conditions, such as diabetes, methods for preventing or delaying the onset of other autoimmune or inflammatory diseases, such as rheumatoid arthritis, methods for preventing or reducing inflammation, and methods for inhibiting leukocyte migration, the methods comprising administering an effective amount of activated Protein C or precursor Protein C.

APC and Protein C

The present invention contemplates the administration of Protein C in either its activated form (APC) or the inactive precursor form. In accordance with one embodiment of the invention, APC or Protein C may be administered in the form of a Protein C polypeptide, optionally together with one or more pharmaceutically acceptable carriers, diluents or adjuvants. Typically, the Protein C polypeptide is human Protein C, more typically recombinant human Protein C. For example an Protein C polypeptide of the invention may have the amino acid sequence as set forth in SEQ ID NO:1. The present invention also contemplates the use of homologues of human Protein C. Homologues include Protein C polypeptides from non-human species.

The APC and/or protein C utilized in the present invention may be obtained by purification from a suitable source (e.g. blood taken from humans or other animals) or produced by standard recombinant DNA techniques such as those well known to person skilled in the art, and described in, for example, Sambrook et al., Molecular Cloning: a Laboratory Manual, Cold Spring Harbor Laboratory Press (the disclosure of which is incorporated herein by reference).

Within the scope of the term “polypeptide” as used herein are fragments and variants thereof.

The term “fragment” refers to a polypeptide sequence that is a constituent on the full-length Protein C. Fragments typically possess qualitative biological activity in common with the full-length Protein C, and may display enhanced or similar quantitative levels of activity when compared with the full-length molecule. Suitable functional fragments may be of approximately 5 to approximately 450 amino acids in length, approximately 5 to approximately 450 amino acids in length, approximately 5 to approximately 200 amino acids in length, approximately 10 to approximately 100 amino acids in length or approximately 10 to approximately 50 amino acids in length. Suitable functional fragments of APC or Protein C may be produced by polypeptide synthesis, by recombinant DNA techniques or by cleavage of purified natural or recombinant APC or Protein C, for example with trypsin or other suitable protease(s).

The term “variant” refers to a substantially similar sequence to that of a native APC or precursor Protein C. Generally, variant polypeptides may share at least 25%, 30%, 35%, 40%, 45%, 50%, 55%, 60%, 65%, 70%, 75%, 80%, 85%, 90%, 95%, 96%, 97%, 98% or 99% sequence identity with a native, wild-type molecule. Further, a variant polypeptide may include an analogue, wherein the term “analogue” means a polypeptide which is a derivative of Protein C, which derivative comprises addition, deletion or substitution of one or more amino acids such that the polypeptide retains substantially the same function as native Protein C. The term “conservative amino acid substitution” refers to a substitution or a placement of one amino acid for another with similar properties within a polypeptide chain. For example, the substitution of the charged amino acid glutamic acid (Glu) for the similarly charged amino acid aspartic acid (Asp) would be a conservative amino acid substitution. Such amino acid substitutions may enhance biological activity or expression of the variant or analogue when compared with native, wild-type Protein C.

The present invention also contemplates the administration of APC or precursor Protein C via gene therapy. Accordingly, embodiments of the present invention also provide for the administration in the form of a polynucleotide encoding a Protein C polypeptide as described above. Typically the polynucleotide encodes human Protein C. For example a Protein C polynucleotide of the invention may have the nucleotide sequence as set forth in SEQ ID NO:2. In addition to polynucleotides encoding the full-length human Protein C, the invention also contemplates the use of polynucleotides encoding homologues of human Protein C and polynucleotides encoding fragments and variants of Protein C. The term “fragment” refers to a nucleic acid sequence that encodes a constituent of the full-length Protein C. Generally, nucleic acid sequence variants encode polypeptides which possess qualitative biological activity in common.

In particular embodiments of the invention a polynucleotide encoding Protein C may be administered in a vector. The vector may be a plasmid vector, a viral vector, or any other suitable vehicle adapted for the insertion and foreign sequences and introduction into eukaryotic cells. Typically the vector is an expression vector capable of directing the transcription of the DNA sequence of a polynucleotide encoding Protein C into mRNA. The vector may include expression control and processing sequences such as a promoter, an enhancer, ribosome binding sites, polyodenylation signals and transcription termination sequences. Examples of suitable viral expression vectors include for example Epstein-barr virus-, bovine papilloma virus-, adenovirus- and adeno-associated virus-based vectors.

Those skilled in the art will also readily appreciate that various modifications may be made to the sequence of the polynucleotide encoding Protein C such that modified variants of the polypeptide are generated in which one or more portions of the encoded polypeptide is replaced by one or more portions of another polypeptide. The portion(s) replaced may correspond to a particular structural or functional domain of the Protein C polypeptide. Such modifications are also included within the scope of the term “variant”. For example, modifications may be made so as to enhance the expression or stability of the Protein C polypeptide or to otherwise increase the effectiveness of the polypeptide to reduce blood glucose levels or prevent or delay the onset of glucose-associated conditions.

Conditions and Diseases

Aspects and embodiments of the present invention find application in the treatment of subjects for the prevention or delay of onset of conditions associated with or characterized by, at least in part, elevated plasma glucose levels (referred to herein as glucose-associated conditions). Administration of APC or precursor Protein C may prevent the onset, prevent the development or delay, slow or otherwise retard the progression of such conditions and their related complications.

Conditions to which methods and compositions of the invention are applicable include, but are not limited to, type I (insulin-dependent) diabetes, type II (non-insulin-dependent) diabetes, gestational diabetes, impaired glucose tolerance, insulin resistance syndrome, hyperglycaemia and postprandial hyperglycaemia. Further, embodiments of the present invention also find application in the prevention or delay of onset of other autoimmune or inflammatory diseases such as those of the musculoskeletal system, the nervous system, the gastrointestinal system, blood and blood vessels, the endocrine system, and the skin.

By way of example, diseases of the musculoskeletal system to which the present invention may be applicable include arthritis such as rheumatoid arthritis, systemic lupus erythematosus, scleroderma, polymyositis, dermatomyositis, spondyloarthropathies such as ankylosing spondylitis, and Sjogren's syndrome; diseases of the nervous system include multiple sclerosis, myasthenia gravis, Guillain-Barre Syndrome, and autoimmune uveitis; diseases of the gastrointestinal system include Crohn's Disease, ulcerative colitis, primary biliary cirrhosis, and autoimmune hepatitis; diseases of the blood and blood vessels include autoimmune hemolytic anemia, pernicious anemia, autoimmune thrombocytopenia, temporal arteritis, vasculitis, Behcet's disease, and anti-phospholipid syndrome; diseases of the endocrine system include Grave's Disease, Hashimoto's thyroiditis, autoimmune oophoritis and orchitis, and autoimmune disease of the adrenal gland; and diseases of the skin include psoriasis, dermatitis herpetiformis, pemphigus vulgaris, and vitiligo.

In one embodiment, the disease is an arthritis, in particular rheumatoid arthritis.

Compositions and Routes of Administration

Embodiments of the present invention contemplate compositions for reducing blood glucose levels in a subject, for preventing the onset of a condition associated with elevated levels of glucose in the blood, for preventing or delaying the onset of an autoimmune or inflammatory disease, and for preventing or reducing inflammation in sufferers of autoimmune or inflammatory diseases.

According to the methods of present invention compounds and compositions may be administered by any suitable route, either systemically, regionally or locally. By way of example, administration according to embodiments of the invention may be achieved by any standard routes, including intracavitary, intravesical, intramuscular, intraarterial, intravenous, subcutaneous, topical, nasal or oral. Intracavitary administration may be intraperitoneal. The particular route of administration to be used in any given circumstance will depend on a number of factors, including the nature of the condition to be treated, the severity and extent of the condition, the required dosage of the particular compound to be delivered and the potential side-effects of the compound. For example, topical administration of compounds or compositions in accordance with the present invention may be appropriate for subjects undergoing surgery to treat the underlying condition, either before, during or after surgery.

In one embodiment the composition may be in a suitable form for intranasal or administration, for example in an aerosol form suitable for intranasal or oral inhalation or in liquid form for administration of drops. For example the composition may be delivered via a pressurised pump inhaler system, via a pump-action spray, or using any other suitable nasal or oral administration means.

In general, suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions.

Aerosol compositions for oral or nasal administration may contain a lubricating agent in addition to a propellant. Suitable lubricating agents and propellants are known to those skilled in the art. For example the lubricating agent may be polysorbate 85 or oleic acid and the propellant may be a chlorofluorocarbon such as Freon or a hydrofluoroalkane such as hydrofluoroalkane-134a. The particle size distribution in the composition may depend on the intended site of absorption in the body, for example via nasal mucosa or alveoli in the lungs.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Liquid forms for oral or nasal administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral or nasal administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Emulsions for oral administration may further comprise one or more emulsifying agents. Suitable emulsifying agents include dispersing agents as exemplified above or natural gums such as guar gum, gum acacia or gum tragacanth.

Some examples of suitable carriers, diluents, excipients and adjuvants for oral or nasal use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

For administration as an injectable solution or suspension, non-toxic parenterally acceptable diluents or carriers can include, Ringer's solution, medium chain triglyceride (MCT), isotonic saline, phosphate buffered saline, ethanol and 1,2 propylene glycol.

Adjuvants typically include emollients, emulsifiers, thickening agents, preservatives, bactericides and buffering agents.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multilamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The effective dose level of the administered compound for any particular subject will depend upon a variety of factors including: the type of condition being treated and the stage of the condition; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis.

Generally, an effective dosage is expected to be in the range of about 0.1 μg to about 100 mg per kg body weight per week; typically, about 1 μg to about 10 mg per kg body weight per week; about 10 μg to about 11000 μg per kg body weight per week; or about 100 μg to about 1000 μg per kg body weight per week, or about 200 μg to about 800 μg per kg body weight per week.

Alternatively, an effective dosage may be up to about 5000 mg/m². Generally, an effective dosage may be in the range of about 10 to about 5000 mg/m², about 10 to about 2500 mg/m², about 25 to about 2000 mg/m², about 50 to about 1500 mg/m², about 50 to about 1000 mg/m², or about 75 to about 600 mg/m².

Further, it will be apparent to one of ordinary skill in the art that the optimal quantity and spacing of individual dosages will be determined by the nature and extent of the condition being treated, the form, route and site of administration, and the nature of the particular individual being treated. Also, such optimum conditions can be determined by conventional techniques.

The present invention will now be further described in greater detail by reference to the following specific examples, which should not be construed as in any way limiting the scope of the invention.

### EXAMPLES

### Example 1

### APC Reduces Blood Glucose Levels of NOD Mice

NOD (non-obese diabetic) mice have a predisposition to insulitis, display elevate plasma glucose levels and spontaneously develop insulin-dependent diabetes. As such they are an excellent animal model for type I diabetes. The ability of APC to influence plasma glucose levels in NOD mice was tested.

Female NOD mice and Balb/c mice were obtained at an age of 4 weeks and allowed to acclimatize to Gore Hill Research Laboratories at Royal North Shore Hospital animal facility for 1 week before treatment. Mice were maintained under specific pathogen-free conditions, and manipulated in accordance with RNSH Human Animal Care and Ethics Committee.

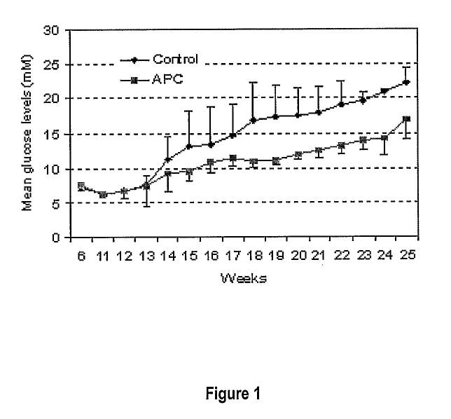

NOD mice were treated with APC intravenously twice a week for 5 weeks commencing at 5 weeks of age. For initial dose response studies, APC was used at 0.5, 1 and 2 mg/kg. Subsequently, mice were treated with 2 mg/kg APC as shown in FIG. 1. As a control group, NOD mice were treated with the same volume of PBS. Blood glucose of both the vehicle and APC groups were monitored.

As seen in FIG. 1, when compared to the control, APC treatment prevented the elevation of glucose levels of NOD mice from week 14, and this reduction was distinct until week 25, when mice were terminated.

### Example 2

### APC Prevents and Delays the Onset of Diabetes in NOD Mice

The ability of APC treatment to prevent or delay the onset of diabetes in NOD mice was assessed from experiments in Example 1. Spontaneous diabetes incidence was monitored until 25 weeks of age. The incidence of diabetes was determined by measurement of blood glucose using Accu-Check Active glucose meter from 10 to 25 weeks of age. Individual mice are classified as diabetic on the basis of positive hyperglycemia (blood glucose>16.7 mM) as previously described (Kawamura et al., 1993).

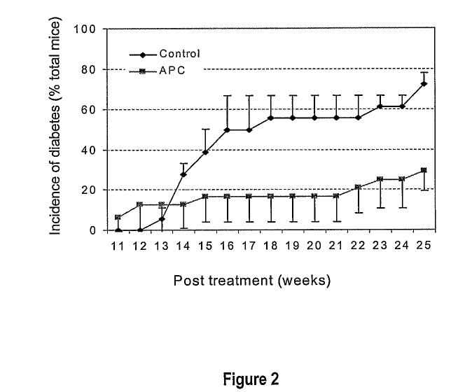

As illustrated in FIG. 2, when NOD mice were pretreated with APC continuously for five weeks, after week 14, there were significantly fewer diabetic mice than in the control (non-APC treated) group. There were 2.5 times more diabetic mice in control groups than in APC treated groups at week 25.

### Example 3

### APC Regulates MMP Production in Splenic T Cells from NOD Mice

Spleen cells from NOD mice or BALB/C mice at 10 week of age were isolated by mincing the organ with the back of a 6-ml syringe in a dish before passing through a 70-μm nylon mesh cell strainer. Cell suspensions were cultured at 10⁶ cells/ml in RPMI 1640 containing 10% (vol/vol) fetal calf serum (FCS). Splenic T cells suspension were added to 24-well plates either alone or with APC. Supernatants were collected at 24 h for gelatin zymography to analyze the production of the gelatinases, MMP-2 and MMP-9.

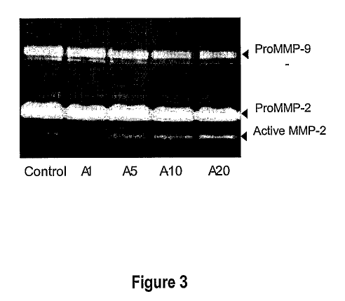

APC inhibited MMP-9 production by splenic T cells in a dose-dependent manner. In contrast, APC dose-dependently stimulated MMP-2 production and activity in these cells (FIG. 3). These results are significant in light of the growing evidence that MMP-9 is pro-inflammatory and MMP-2 is anti-inflammatory. MMP-9 can activate cytokines to exacerbate an inflammatory response. Increased MMP-9 activity has been found to be associated with endothelial dysfunction and apoptosis in a murine model of diabetes (Camp et al., 2003) and in human type 1 diabetic complications (Diamant et al., 2001; Wall et al., 2002). In contrast, lower levels of MMP-2 expression and activation are found in type 1 diabetes and its complications (Hayashi et al., 2003) (Singh et al., 2001). MMP-2 can inhibit inflammation by truncating and inactivating monocyte chemoattractant protein (MCP)-3, which inhibits the infiltration of monocytes.

### Example 4

### APC Inhibits Human Leukocyte Migration

The inventors have also studied the effect of APC on the migration of leukocytes from patients with type I diabetes.

Human monocytes and neutrophils were isolated from heparinized blood collected from type I diabetic patients and normal volunteers using Optiprep medium (Sigma) and Mono-Poly Resolving Medium (ICN Biomedicals Inc). Cells were resuspended in RPMI 1640 medium (JRH Biosciences) containing 2% (w/v) bovine serum albumin. The migration of monocytes and neutrophils in response to APC treatment was detected by a chemotaxis assay using plastic modified Boyden chambers described previously (Thakur and Willcox, 1998).

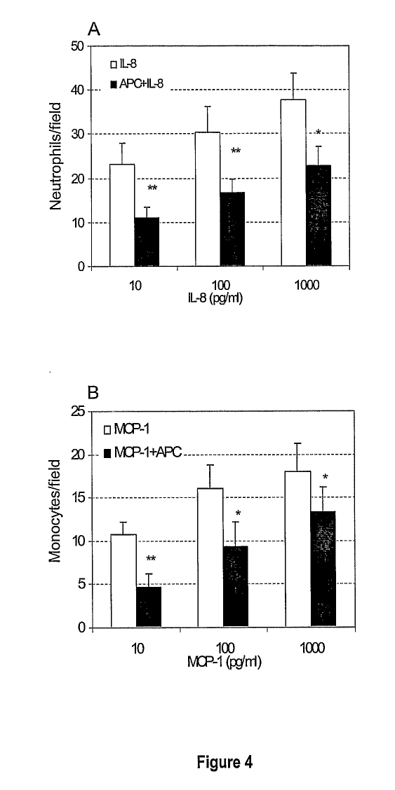

APC decreased IL-8-induced neutrophil migration (FIG. 4) and macrophage chemoattractant protein (MCP)-1-induced monocyte migration in vitro (FIG. 4B). Leukocyte migration is a key feature of inflammation and is characteristic of the changes seen in the development and progression of diabetes. These results suggest that APC can beneficially modulate the function of leukocytes to prevent migration induced by chemotactic agents, implicating APC in a dampening of infiltration and in reducing the inflammatory response.

### Example 5

### APC Inhibits MMP-9 Production in Human Rheumatoid Arthritis Patients

As MMP-9 has been implicated in the development and progression of inflammatory joint diseases such as rheumatoid arthritis, the inventors then investigated the role of APC in the regulation of MMP-9 activity in monocytes isolated from patients with rheumatoid arthritis (RA). Whole blood samples (20 ml/patient) were obtained from 19 patients with RA in accordance with ethics approval by the ethical committee of Royal North Shore Hospital (RNSH). All patients fulfilled the American College of Rheumatology criteria for RA. All participants gave their written informed consent. Whole blood was collected into tubes containing EDTA. Monocytes were isolated using Optiprep (Sigma, St. Louis Mo.) according to manufacturer's instructions. Viability of monocytes was determined by trypan blue exclusion test. Purity of monocytes was determined using anti-CD68 antibody (DAKO Corporation, Carpinteria Calif.) immunostaining. Cells were used for further experiments if both viability and purity were above 95%.

Cells were cultured in RPMI-1640 containing 10% FCS and supplemented with 2 mM L-glutamine, 100 U/ml penicillin and 100 μg/ml streptomycin. Before treatment, cells were pre-incubated in RPMI-1640 with 2% MMP-free serum (basal medium) for 4 hours.

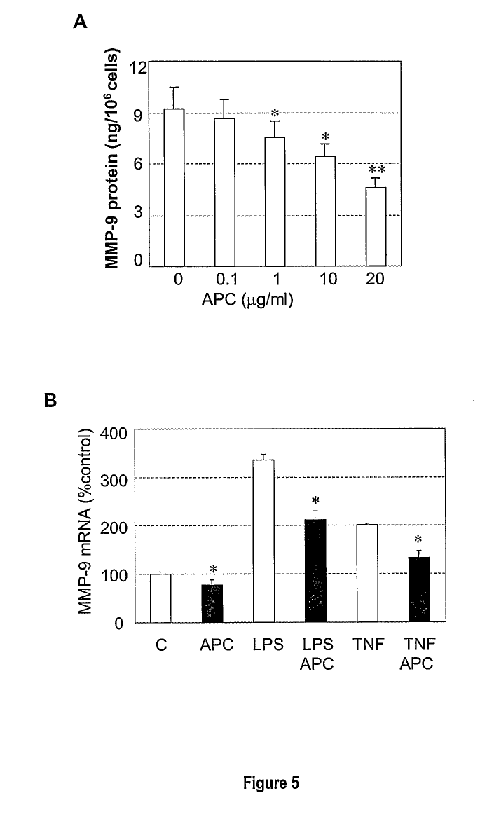

Monocytes were treated with APC for 24 hours and culture supernatants were assessed for MMP-9 by ELISA (R & D Systems, Inc., Minneapolis, Minn., according to manufacturer's instructions) and gelatin zymography (as described in Herron et al., 1986). ELISA results showed that cells from RA patients produced relatively high levels of MMP-9 (>9 ng/10⁶ cells) which was dose-dependently inhibited by 0.1-20 μg/ml APC (FIG. 5A). At 20 μg/ml APC reduced the levels of secreted MMP-9 by approximately 50%. The cell line Mono Mac6 (Mac6), which is characteristic of mature monocytes, was also tested. In Mac6 cells, APC significantly reduced the low basal levels of MMP-9 as well as TNF-α- or LPS-stimulated MMP-9 present in culture supernatants (data not shown).

To determine the mechanism of action, the inventors investigated whether APC might degrade MMP-9 in monocyte supernatants. Mac6 cells were stimulated with TNF-α for 24 hours and cell-free medium containing MMP-9 was collected and incubated with APC for a further 24 hours. Zymography showed that there was no degradation of MMP-9 in response to APC, whereas activation of MMP-2 was distinct (data not shown). This indicates that the reduction of MMP-9 caused by APC is not by a direct degradation of MMP-9.

To detect whether APC inhibited MMP-9 gene expression in monocytes, Mac6 cells were treated with APC for 2 hours and MMP-9 was detected by real time PCR. Total RNA was extracted using Tri Reagent (Sigma) according to the manufacturer's instructions. Single stranded cDNA was synthesized from total RNA using AMV reverse transcriptase (RT) and Oligo (dT)₁₅ as a primer (Promega Corp., Madison, Wis.). PCR was carried out and MMP-9 mRNA semi-quantified on a Rotor-gene 3000A (Corbett Research, Sydney, NSW, Australia). Samples were normalized to the housekeeping gene RPL13A and results reported relative to the control. Primers used were as follows: MMP9 (90 bp) sense primer 5′ CTGGGCAGATTCCAAACCT (SEQ ID NO:3); anti-sense primer 5′ CGGCAAGTCTTCCGAGTAGT (SEQ ID NO:4); RPL13A (152 bp) sense primer 5′ AAGCCTACAAGAAAGTTTGCCTATC (SEQ ID NO:5); anti-sense primer 5′ TGTTTCCGTAGCCTCATGAGC (SEQ ID NO:6). When used at 20 μg/ml, APC reduced MMP-9 gene levels by approximately 20%, 35% or 40% compared to control, TNF-α or LPS-stimulated cells, respectively (FIG. 5B).

### Example 6

### APC Inhibits NF-κB Activation in Human Rheumatoid Arthritis Patients Via EPCR

The transcription factor NF-κB is a crucial factor involved in the up-regulation of proinflammatory cytokines by monocytes. The inventors therefore tested whether APC regulated the activity of NF-κB in RA monocytes.

Cells were incubated with APC for 24 hours in the presence or absence of an inflammatory stimulus (TNF-α or LPS) and whole cell lysates were subjected to western blotting using an antibody which recognizes the active form of NF-κB. Cells were incubated for 30 minutes at 4° C. before sonicating for 1 minute at 4° C. The sample was centrifuged at 16,000 g for 15 minutes and protein concentration was determined using the Bradford protein assay (Bio-Rad, Hercules, Calif.). Equally loaded protein was separated by 10% SDS-PAGE and electrotransferred onto Immobion™ Transfer membranes (PVDF) (Millpore Corporation, Bedford, Mass.). Membranes were blocked with 5% BSA in TBST (50 mM, Tris, pH 7.6, 150 mM NaCl, 0.1% Tween 20) for 1 hour and incubated overnight with mouse anti-human monoclonal antibodies to the active form of NF-κB (1 μg/ml, Chemicon International, Inc., Temecula, Calif.) in TBS containing 1% BSA and then washed with TBST. Following the incubation with a horseradish peroxidase-conjugated secondary antibody for 1 hour, immunoreactivity was detected using the ECL detection system (Amersham, Piscataway, N.J.).

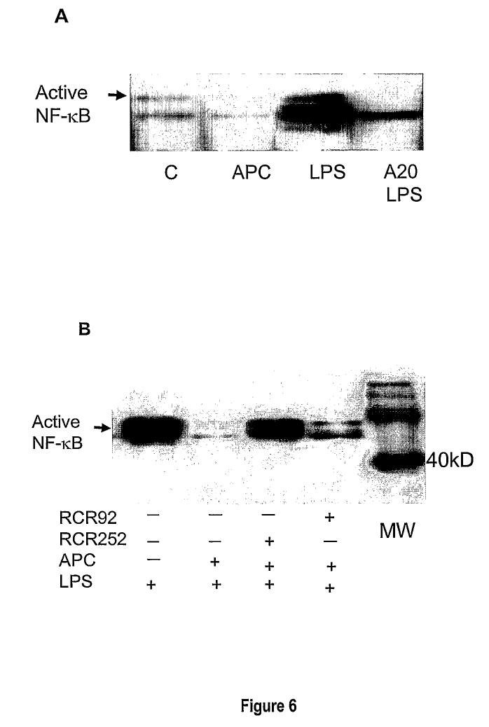

Activated NF-κB was present in RA monocytes under basal conditions and was enhanced by LPS (FIG. 6A). APC (20 μg/ml) markedly inhibited the active form of NF-κB particularly in LPS stimulated cells.

EPCR is a specific receptor for APC and mediates many of its anti-inflammatory effects. To determine whether the effect of APC on NF-κB is EPCR dependent, RA monocytes cells were treated with two anti-EPCR antibodies, RCR252 (a blocking antibody to EPCR) and RCR92 (a non-blocking antibody) prior to APC treatment. Cell lysates were used for analyse the activity of NF-κB. APC's inhibitory effect on LPS-induced NF-κB activity was almost totally reversed by RCR252, whereas RCR92 had only limited effect (FIG. 6B).

### Example 7

### APC Inhibits Inflammation in a Mouse Model of Arthritis

To generate a mouse model of arthritis, DBA/J1 male mice were injected intradermally with chicken type II collagen (100 μg collagen/mouse). Type II collagen was injected again on day 21 to boost the onset of arthritis.

After the first collagen injection, mice received either 2 mg/kg APC or PBS (control) (8 mice/group) via intraperitoneal injection until the development of arthritis, One week after boost, mice were examined for clinical signs of arthritis. The presence of arthritis was determined by examining the appearance of the front and hind paws. Severity was graded for each paw using an established scoring system: 0=no swelling, 1=swelling in one digit or mild edema, 2=several digits and moderate swelling, 3=severe swelling affecting most digits, and 4=the most severe swelling. An arthritis score was assigned to each mouse by summing the scores of each paw.

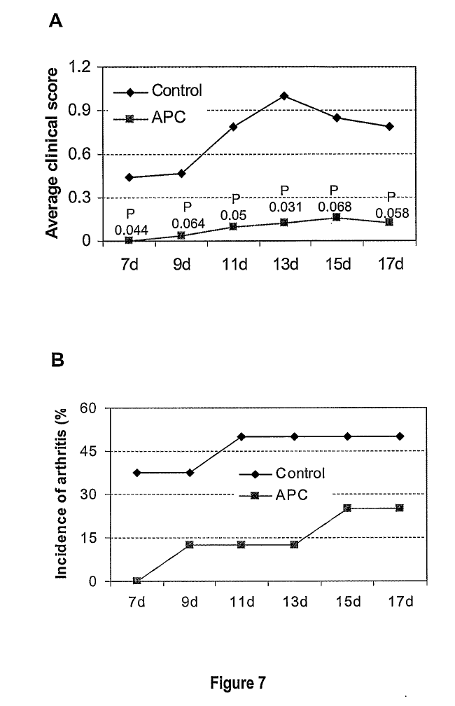

As shown in FIG. 7, mice treated with APC exhibited a marked reduction in the incidence and severity of symptoms of arthritis compared to the control mice, treated with PBS. The APC treatment did not have any impact on the weight of the mice.)

### Example 8

### Compositions for Treatment

In accordance with the best mode of performing the invention provided herein, specific preferred compositions are outlined below. The following are to be construed as merely illustrative examples of compositions and not as a limitation of the scope of the present invention in any way.

### Example 8(A)

### Composition for Inhalation Administration

For an aerosol container with a capacity of 20-30 ml: a mixture of 10 mg of APC with 0.5-0.8% by weight of a lubricating agent, such as polysorbate 85 or oleic acid, is dispersed in a propellant, such as freon, and put into an appropriate aerosol container for either intranasal or oral inhalation administration.

### Example 8(B)

### Composition for Oral Administration

A composition of a suitable agent in the form of a capsule may be prepared by filling a standard two-piece hard gelatin capsule with 500 mg of APC, in powdered form, 100 mg of lactose, 35 mg of talc and 10 mg of magnesium stearate.

### Example 8(C)

### Composition for Parenteral Administration

A composition for parenteral injection could be prepared to contain 0.05 mg to 5 g of APC in 10 mls to 2 litres of 1% carboxymethylcellulose.

Similarly, a composition for intravenous or intra-articular infusion may comprise 250 ml of sterile Ringer's solution, and 0.05 mg to 5 g of APC.

### Example 8(D)

### Composition for Topical Administration

A typical composition for delivery as a topical ointment includes 1.0 g of APC, together with white soft paraffin to 100.0 g, dispersed to produce a smooth, homogeneous product.

### REFERENCES

Atkinson and Maclaren, 1994. The pathogenesis of insulin-dependent diabetes mellitus. N. Engl. J Med 331, 1428-1436.
Camp et al., 2003. Gelatinase B(MMP-9) an apoptotic factor in diabetic transgenic mice. Diabetologia.
Diamant et al., 2001. Elevated matrix metalloproteinase-2 and -9 in urine, but not in serum, are markers of type 1 diabetic nephropathy. Diabet Med 18, 423-424.
Esmon et al. 1997. The protein C pathway: new insights. Thromb Haem 78, 70-74.
Gale et al. 2004. European Nicotinamide Diabetes Intervention Trial (ENDIT): a randomized trial of intervention before the onset of type 1 diabetes. Lancet 363, 925-931.
Giannelli et al., 2004. MMP-2, MMP-9, TIMP-1 and TIMP-2 levels in patients with rheumatoid arthritis and psoriatic arthritis. Clin Exp Rheumatol 22, 335-338.
Hayashi et al., 2003. Angiotensin II receptor blockade prevents microangiopathy and preserves diastolic function in the diabetic rat heart. Heart 89, 1236-1242.
Herron et al. 1986. Secretion of metalloproteinases by stimulated capillary endothelial cells. II. Expression of collagenase and stromelysin activities is regulated by endogenous inhibitors. J Biol Chem 261, 2814-2818
Huck et al. 2001. HLA-DR53 molecules restrict glutamic acid decarboxylase peptide presentation to T cells of a Type I diabetes patient: specification of the trimolecular HLA-peptide/T-cell receptor complex. Diabetologia 44, 70-80.
Jovanovic et al., 2000. Stimulation of 92-kd gelatinase (matrix metalloproteinase 9) production by interleukin-17 in human monocytelmacrophages: a possible role in rheumatoid arthritis. Arthritis Rheum 43, 1134-1144.
Joyce et al., 2001. Gene expression profile of antithrombotic protein C defines new mechanisms modulating inflammation and apoptosis. J Biol Chem. 276, 11199-11203.
Kawamura et al., 1993. Prevention of autoimmune type I diabetes by CD4+ suppressor T cells in superantigen-treated non-obese diabetic mice. J. Immunol. 151, 4362-4370.
Singh et al., 2001. High glucose decreases matrix metalloproteinase-2 activity in rat mesangial cells via transforming growth factor-beta1. Exp. Nephrol. 9, 249-257.
Thakur and Willcox, 1998. Cytokine and Lipid Inflammatory Mediator Profile of Human Tears During Contact Lens Associated Inflammatory Diseases*1. Experimental Eye Research 67, 9-19.
Wall et al., 2002. Differential expression of matrix metalloproteinases during impaired wound healing of the diabetes mouse. J Invest Dermatol. 119, 91-98.

1. A method for preventing the elevation of blood glucose levels in a subject, the method comprising administering to the subject an effective amount of activated Protein C or precursor Protein C.

2. The method of claim 1 wherein the Protein C is a polypeptide comprising the amino acid sequence as set forth in SEQ ID No. 1.

3. The method of claim 1 wherein the Protein C is administered in the form of a nucleic acid molecule encoding Protein C.

4. The method of claim 3 wherein the nucleic acid comprises a nucleotide sequence as set forth in SEQ ID No. 2.

5. The method of claim 4 wherein the nucleotide sequence is located in a nucleic acid construct operably linked to a promoter active in the subject to be treated.

6. A method for preventing or delaying the onset of an autoimmune or inflammatory disease in a subject, the method comprising administering to the subject an effective amount of activated Protein C or precursor Protein C.

7. The method of claim 6 wherein the autoimmune or inflammatory disease is selected from the group consisting of: hyperglycaemia, postprandial hyperglycaemia, insulin resistance, type I diabetes, type II diabetes, gestational diabetes, and arthritis or other inflammatory joint disease.

8. The method of claim 7 wherein the autoimmune or inflammatory disease is an arthritis.

9. The method of claim 8 wherein the arthritis is rheumatoid arthritis.

10. A method for preventing or reducing inflammation in a subject suffering from an autoimmune or inflammatory disease, the method comprising administering to the subject an effective amount of activated Protein C or precursor Protein C.

11. A method for preventing or delaying the onset of a glucose-associated condition in a subject, the method comprising administering to the subject an effective amount of activated Protein C or precursor Protein C.

12. The method of claim 11 wherein the glucose-associated condition is selected from the group consisting of: hyperglycaemia, postprandial hyperglycaemia, insulin resistance, type I diabetes, type II diabetes, and gestational diabetes.

13. The method of claim 12 wherein the glucose-associated condition is type I or type II diabetes.

14. A method for preventing or delaying the onset of diabetes in a subject, the method comprising administering to the subject an effective amount of activated Protein C or precursor Protein C.

15. The method of claim 14 wherein the diabetes is type I diabetes, type II diabetes or gestational diabetes.

16. A method for inhibiting the migration of leukocytes in a subject, the method comprising administering to the subject an effective amount of activated Protein C or precursor Protein C.

17. The method of claim 16 wherein the leukocytes are neutrophils or monocytes.

18. A method as claimed in any one of the preceding claims wherein the protein or nucleic acid construct is administered in the form of a composition together with one or more pharmaceutically acceptable carriers, adjuvants or diluents.

19. A composition when used for preventing the elevation of blood glucose levels, the composition comprising activated Protein C or precursor Protein C together with one or more pharmaceutically acceptable carriers, diluents or adjuvants.

20. A composition when used for preventing or delaying the onset of an autoimmune or inflammatory disease, the composition comprising activated Protein C or precursor Protein C together with one or more pharmaceutically acceptable carriers, diluents or adjuvants.

21. A composition when used for preventing or reducing inflammation in a subject suffering from an autoimmune or inflammatory disease, the composition comprising activated Protein C or precursor Protein C together with one or more pharmaceutically acceptable carriers, diluents or adjuvants.

22. A composition when used for preventing or delaying the onset of a glucose-associated condition, the composition comprising activated Protein C or precursor Protein C together with one or more pharmaceutically acceptable carriers, diluents or adjuvants.

23. A composition when used for preventing or delaying the onset of diabetes, the composition comprising activated Protein C or precursor Protein C together with one or more pharmaceutically acceptable carriers, diluents or adjuvants.

24. A composition when used for inhibiting migration of leukocytes, the composition comprising activated Protein C or precursor Protein C together with one or more pharmaceutically acceptable carriers, diluents or adjuvants.

25. Use of activated Protein C or precursor Protein C for the manufacture of a medicament for the prevention of elevation of blood glucose levels in a subject.

26. Use of activated Protein C or precursor Protein C for the manufacture of a medicament for preventing or delaying the onset of an autoimmune or inflammatory disease in a subject.

27. Use of activated Protein C or precursor Protein C for the manufacture of a medicament for preventing or delaying the onset of a glucose-associated condition.

28. Use of activated Protein C or precursor Protein C for the manufacture of a medicament for preventing or delaying the onset of diabetes.


SEQUENCE LISTING (6 sequences)

SEQ ID NO: 1 - protein, 461 aa, Homo sapien
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - DNA, 1386 bp, Homo sapien
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 3 - DNA, 19 bp, Artificial Sequence
  Synthetic sequence
ctgggcagattccaaacct

SEQ ID NO: 4 - DNA, 20 bp, Artificial Sequence
  Synthetic sequence
cggcaagtcttccgagtagt

SEQ ID NO: 5 - DNA, 25 bp, Artificial Sequence
  Synthetic sequence
aagcctacaagaaagtttgcctatc

SEQ ID NO: 6 - DNA, 21 bp, Artificial Sequence
  Synthetic sequence
tgtttccgtagcctcatgagc